(19) United States
(12) Patent Application Publication
(10) Pub. No.: US 2003/0031626 A1
(43) Pub. Date: Feb. 13, 2003

(54) Process for the production of pharmaceutical preparations

(76) Inventors: Thomas Rheinlander, Liestal (CH); Gul Caglar, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/207,377

(22) Filed: Jul. 26, 2002

Related U.S. Application Data
(60) Provisional application No. 60/309,206, filed on Aug. 2, 2001.

(30) Foreign Application Priority Data
Jul. 26, 2001 (DE) 10137512.8

(51) Int. Cl. A61B 8/00

(52) U.S. Cl. 424/9.5

(57) ABSTRACT

The invention relates to a new process for the production of pharmaceutical preparations or intermediate products thereof, whereby the pharmaceutical preparation is present as a dispersion, the particles that are present in the dispersion have a charge distribution, and at least a portion of the particles that are present in the dispersion are separated with the aid of ion exchangers or by electrophoretic separation processes. In addition, devices for performing the process are disclosed. Pharmaceutical preparations that can be obtained with the aid of the process as well as use thereof are also described.

### CROSS-REFERENCE TO RELATED APPLICATION

This application claims the benefit of the filing date of U.S. Provisional Application Serial No. 60/309,206 filed Aug. 2, 2001.

The invention relates to a new process for the production of pharmaceutical preparations or intermediate products thereof, whereby the pharmaceutical preparation is present as a dispersion, the particles that are present in the dispersion have a charge distribution, and at least a portion of the particles that are present in the dispersion are separated with the aid of ion exchangers or by electrophoretic separation processes. In addition, devices for performing the process are disclosed. Pharmaceutical preparations that can be obtained with the aid of the process as well as use thereof are also described.

Pharmaceutical preparations can be present in the form of dispersions, e.g., as parenteral fat emulsions or crystal suspensions. In nuclear resonance tomography, magnetite dispersions are used as contrast media (see, e.g., EP 186 616, U.S. Pat. Nos. 5,328,681, US 5,424,419, US 5,766,572). In International Application WO 98/05430, a process for separating magnetic materials from pharmaceutical preparations as well as agents that are produced according to this process are described. It is shown that magnetite dispersions, in which certain magnetic particles are selected, are especially well suited for magnetic resonance angiography.

In addition, it is already known that ions up to high-molecular biomolecules, such as proteins, can be separated by ion exchange or electrophoresis (Schwedt, Analytische Chemie [Analytical Chemistry], Thieme Verlag, 1995, 301 ff+365 ff).

In engineering, however, particles are pre-charged by ion bombardment, capacitive induction or contact electrification for electrostatic or electrophoretic separation of minerals, contaminants, valuable products, etc. (Bronkala, Ultmann's Encyclopedia of Industrial Chemistry (5th Ed.) B2, 20-1, VCH Weinheim 1990). The separation of particles in the micrometer range is carried out dry, while it is done wet in the nanometer range. With the dielectrophoresis, however, in principle all compounds, even uncharged, can be separated because of their polarizability with electric steady fields as well as alternating fields.

For special applications, the use of ion exchangers in connection with pharmaceutical agents is proposed. Ion exchangers in a separate container are thus proposed for changing the pH of pH-sensitive pharmaceutical solutions from their storage pH to a suitable administration pH (WO 9823375). In addition, for the delayed release of active ingredients such as pharmaceutical agents, ion-exchange resins are proposed that are charged with active ingredient and also are covered or charged with an oppositely charged polymer (DE 19619313).

The object of this invention is to provide another process with whose aid the production of pharmaceutical preparations from dispersions is accomplished.

This object is achieved with the new process for the production of pharmaceutical preparations according to claim 1.

The new production process separates dispersions not with the aid of a magnetic filter, but rather with the aid of ion exchangers or by electrophoresis.

In contrast to the process that is known from WO 98/05430, dispersions in the process according to the invention are not separated by magnetic forces but rather by electrostatic forces. The process is therefore not limited to magnetic materials like the process that is described in WO 98/05430. The process according to the invention, however, requires that the particles that are to be selected be electrostatically charged.

The charge of the particles contributes to the stabilization of the dispersion and is therefore present in most cases or is a desired effect. It originates from bonded or adsorbed ions, amino acids, proteins, lipids, lipoproteins, nucleotides, ribonucleic acids, deoxyribonucleic acids, carbohydrates, glycoproteins, natural or synthetic polymers as well as derivatives thereof, activated carbon, silicon compounds and/or surface-active substances such as surfactants.

The particles can be combined or are combined with structure-specific substances that also have a partially stabilizing effect. Such structure-specific substances are, i.a., antibodies, antibody fragments, agonists that bind specifically to receptors, such as cytokines, lymphokines, endothelins or antagonists thereof, other specific peptides or proteins, receptors, enzymes, enzyme substrates, nucleotides, ribonucleic acids, deoxyribonucleic acids, carbohydrates or lipoproteins. As structure-specific substances, those are preferred whose binding constant is in the range of 10⁵-10¹⁵ l/mol. The structure-specific substances can be labeled with the particles with the aid of familiar processes. An alternative is the binding via antibodies that are directed against the surface of the particles, e.g., against the shell material.

In contrast to biochemical molecules such as proteins, where all molecules of a protein have the same charge under the same conditions, particles in pharmaceutical preparations in the form of dispersions, for example in magnetite dispersions or ultrasonic contrast medium dispersions, have a charge distribution, i.e., ions with a different number of elementary charges (single, double, triple-charged, etc.) are present side by side in the dispersion. The separation of such dispersions with the aid of the inventive process therefore results in new pharmaceutical preparations that have an altered charge distribution. Since the receiving of particles introduced parenterally in the human or the animal in the monocytic phagocyte system (MPS) or in other body parts depends on, i.a., their charge, the separation according to the charge also allows an influencing control on the in-vivo pharmacokinetic properties of the pharmaceutical preparations.

Particles in magnetite dispersions are magnetic, and particles in ultrasonic contrast medium dispersions are generally filled with a gas.

The particles that are to be selected advantageously have a size of less than 10 μm. Especially preferred are particle sizes of 1 to 100 nm.

A device that is suitable for performing the process according to the invention consists of a separation chamber, which contains an ion exchanger and has an inlet and outlet. Special embodiments of such a device are shown in

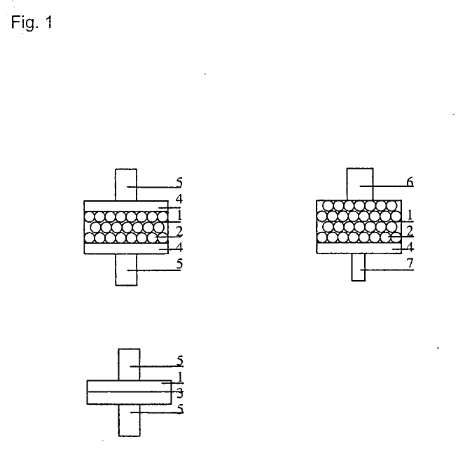

FIG. 1. In this case, (1) refers to a separation chamber, (2) refers to ion exchange particles, (3) refers to an ion-exchange membrane, (4) refers to a frit or a filter, and in each case (5) refers to a connection, (6) to the inlet and (7) to the outlet.

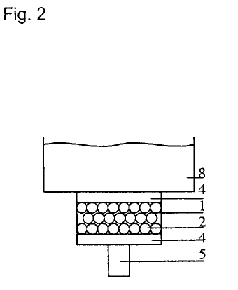

FIG. 2 diagrammatically shows a device that is integrated in an infusion instrument. In this case, (8) is to refer to the infusion container.

The device can also be designed as an attachment filter for an infusion or injection instrument.

As ion exchangers, all standard commercially available ion exchangers are considered. The most common ion exchangers are gel-like, whereby the ions must diffuse through the gel to the exchanger groups with a porosity of, for example, 3 nm. Macroporous ion exchangers can also be used that have pores in the range of about 100 nm. Preferred are ion exchangers, in which the exchanger groups rest on tentacles. In addition, ion-exchange membranes can also be used. Weak/strong ion exchangers are also provided. Weak ion exchangers contain weak acid or base groups, such as, e.g., R—COOH; strong ion exchangers contain strong acid or base groups such as, e.g., R—SO₃⁻. For complete removal of all charged compounds, the use of anion and cation exchangers is necessary.

The adjustment of a specific pH for the separation can be advantageous. In addition, it is advantageous to select the counterions of the ion exchangers and the displacement salts or an optionally used electrophoresis buffer in such a way that the ions and salts are physiologically compatible and can remain in the products.

With the electrophoretic separation process, electrophoresis can be done as free electrophoresis or carrier electrophoresis, such as, e.g., gel and paper electrophoresis.

The process according to the invention is especially suitable for the production of contrast medium dispersions, such as, e.g., magnetic resonance or ultrasonic contrast medium dispersions. In this case, already present magnetic resonance dispersions or ultrasonic contrast medium dispersions are treated with the process according to the invention. By the selection of certain particles, the physical properties of the contrast medium dispersions are altered. The dispersions that are thus altered can be used for certain diagnostic problems (e.g., nuclear resonance angiography).

The process for the separation of disruptive foreign particles from pharmaceutical preparations is also suitable, moreover.

The examples below explain the invention without intending that they be limited to the latter.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLE 1

Separation of Charged Particles from a Dispersion of Charged Particles by Means of Electrophoresis

Ultrafiltered magnetite suspension (produced according to U.S. Pat. No. 4,101,435, Example 7) in 10 mmol of sodium acetate buffer pH 5 [zeta potential: −27 mV, 70 nm particle diameter (photon correlation spectroscopy (PCS)), r1: 20 l/mmol of Fe/s, r2: 160 l/mmol of Fe/s (magnetic resonance (MR)), 5 mT/mol of Fe (magnetic relaxometry (MRX) according to DE 19503664, solid), 1.1 mT/mol of Fe (liquid MRX)] is dropped onto the Macherey-Nagel paper MN 866 facing away from the anode. At 20 V/cm, a portion of the particles is deflected to the anode, so that at the end of the travel distance, the particles can be collected at different outlets. A fraction that is hardly deflected (−23 mV, 60 nm, 5.1/0.7 mT/mol) as well as a fraction that is deflected to the anode (−25 mV, 60 nm, 5.4/0.8 mT/mol) are obtained.

### EXAMPLE 2

Separation of Charged Particles from a Dispersion of Charged Particles with a Strong Ion Exchanger

1 ml of desalinated strong tentacle anion exchanger Fractogel EMD TMAE 650 S by Merck is added to 5 ml of ultrafiltered magnetite suspension [zeta potential: −34 mV, 70 nm particle diameter (PCS), r1: 20 l/mmol/s, r2: 160 l/mmol/s (MR), 5 mT/mol (solid MRX), 1.1 mT/mol (liquid MRX)], diluted with distilled water and shaken for 1 hour. The supernatant is dialyzed (−35 mV, 76 nm, 22/167 l/mmol/s, 5.2/1.1 mT/mol). The exchanger beads are shaken overnight with 1 M NaCl, and the supernatant is dialyzed (−16 mV, 67 nm, 18/69 l/mmol/s, 1.2/0.3 mT/mol).

### EXAMPLE 3

Ion-Exchange Attachment

In a container with two connections and at least one filter frit (see FIG. 1), suitable ion exchanger is filled. If the ion exchanger is present suspended in a salt solution, the solution is then replaced by flushing with water.

### EXAMPLE 4

Separation of Charged Particles from a Dispersion by Means of an Ion-Exchange Attachment

On PCS, 50 μl of a negatively charged latex suspension in 10 ml of distilled water produces a counting rate of 615 kCps. 2 ml of this solution is added to an ion-exchange attachment according to Example 3, filled with 1 ml of weak tentacle anion exchanger Fractogel EMD DMAE 650 S by Merck and flushed with distilled water. The passage still has only a counting rate of 0.4 kCps. This reflects the separation of the charged particles.

### EXAMPLE 5

Separation of Charged Particles from a Dispersion of Charged Particles with an Ion-Exchange Attachment

1 ml of magnetite suspension [zeta potential: −31 mV, 66 nm particle diameter (PCS), r1: 20 l/mmol/s, r2: 160 l/mmol/s (MR), 5 mT/mol (solid MRX), 1.1 mT/mol (liquid MRX)] is added to an ion-exchange attachment according to Example 4. Then, it is flushed with distilled water until the collected discharge is no longer brown. The discharge is dialyzed (−30 mV, 66 nm, 24/170 l/mmol/s, 7/1, 7 mT/mol).

Then, it is flushed with 10 mmol of NaOH (pH 12), and the discharge is collected. The latter is neutralized with HCl and dialyzed (−21 mV, 52 nm, 19/80 l/mmol/s, 1.9/0.3 mT/mol).

The entire disclosure of all applications, patents and publications, cited herein and of corresponding German Priority Application No. 101 375 12.8, filed Jul. 26, 2001, and U.S. Provisional Application Serial No. 60/309,206, filed Aug. 2, 2001 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for the production of pharmaceutical preparations or intermediate products thereof, whereby the pharmaceutical preparation is present as a dispersion, the particles that are present in the dispersion have a charge distribution, and at least a portion of the particles that are present in the dispersion are separated with the aid of ion exchangers or by an electrophoretic separation process.

2. Process according to claim 1, characterized in that the particles are magnetic particles.

3. Process according to claim 1, wherein the particles are gas-filled particles.

4. Process according to claim 1, wherein the particles are disruptive foreign particles.

5. Process according to one of the preceding claims, wherein the particle size of the particles to be selected is less than 10 μm.

6. Device for performing the process according to claim 1, wherein the device consists of a separation chamber, which contains an ion exchanger and has an inlet and outlet.

7. Device according to claim 6, wherein the ion exchanger is a strong or weak anion exchanger and/or cation exchanger.

8. Device according to claim 6 or 7, wherein the ion exchanger is a tentacle ion exchanger.

9. Device according to one of claims 6 to 8, wherein the ion exchanger is an ion-exchange membrane.

10. Device according to one of claims 6 to 9, wherein the ion exchanger with physiologically compatible counterions has been documented previously or the displacement of adsorbed ions is carried out with physiologically compatible salts.

11. Device according to one of claims 6 to 10, wherein it is integrated in an injection or infusion instrument.

12. Device according to one of claims 6 to 10, wherein it is designed in the form of an attachment filter for an infusion or injection instrument.

13. Device according to one of claims 6 to 12, wherein the device is sterile.

14. Pharmaceutical preparations that can be obtained by a process according to claim 1.

15. Contrast media for magnetic resonance diagnosis that can be obtained by treatment of magnetic particle suspensions according to the process of claim 1.

16. Contrast media for ultrasonic diagnosis that can be obtained by treatment of ultrasonic contrast medium suspensions according to the process of claim 1.

17. Use of preparations according to claim 14 as contrast media.
